(19) United States
(12) Patent Application Publication
(10) Pub. No.: US 2023/0355166 A1
(43) Pub. Date: Nov. 9, 2023

(54) ARTIFICIAL INTELLIGECE-BASED POSTURE DISCRIMINATION DEVICE USING BODY PRESSURE SENSORS AND METHOD THEREOF

(71) Applicant: Ninebell Healthcare Co., Ltd., Seongnam-si (KR)

(72) Inventors: Young Dae LEE, Seoul (KR); Jie Young CHOI, Seongnam-si (KR); Kun Chang LEE, Goyang-si (KR); Min Gyeong KIM, Seoul (KR); Francis Joseph COSTELLO, Seoul (KR)

(73) Assignee: Ninebell Healthcare Co., Ltd., Seongnam-si (KR)

(21) Appl. No.: 18/299,824

(22) Filed: Apr. 13, 2023

(30) Foreign Application Priority Data
May 4, 2022 (KR) 10-2022-0055253

(51) Int. Cl. A61B 5/00; G06N 3/0475; G06N 3/045

(52) U.S. Cl. CPC A61B 5/447; A61B 5/4561; A61B 5/6892; A61B 5/7264; A61B 5/0002; G06N 3/0475; G06N 3/045; A61B 2562/0247

(57) ABSTRACT

An artificial intelligence-based posture discrimination device using body pressure sensors and a method thereof are proposed. The device includes a body pressure sensor module configured to measure body pressure of a user, touching the frame of the bed or the mattress, by using a plurality of body pressure sensors, a sample body pressure distribution data generation module configured to learn and generate the corresponding user's sample body pressure distribution data by using a Generative Adversarial Network (GAN), and a posture discrimination module configured to analyze the corresponding user's actual body pressure distribution data, and discriminate the corresponding user's lying postures after learning and predicting the time-series body pressure distribution data in the two-dimensional format by using an ensemble artificial intelligence deep learning technique, so that as the user's lying postures are more accurately discriminated, the user's postures may be effectively changed, thereby increasing pressure ulcer prevention functionality and convenience.

### CROSS REFERENCE TO RELATED APPLICATION

The present application claims priority to Korean Patent Application No. 10-2022-0055253, filed May 4, 2022, the entire contents of which is incorporated herein for all purposes by this reference.

### FIELD OF THE DISCLOSURE

The present disclosure relates to an artificial intelligence-based posture discrimination device using body pressure sensors and a method thereof.

### BACKGROUND OF THE DISCLOSURE

In general, changing a patient's body position effectively by discriminating user's lying postures is related to increasing pressure ulcer prevention functionality and convenience.

In particular, the discriminating of the user's lying postures is expected to provide convenience to medical personnel and care givers who carry out tasks of periodically changing body positions of immobilized patients in medical environments and at home. In fact, medical bed-related technology is one of the important clinical medical technologies, and is considered essential for building advanced medical environments.

As medical service conditions have improved due to the development of economy and medical technology, people's average life expectancy has been extended, so the proportion of bedridden patients in geriatric facilities and medical institutions is increasing, and for this reason, responsibilities of health care managers are emphasized for maintaining patients' health and quality of life.

Since the increase in the elderly population and long-term hospitalized patients is directly related to the increase in social costs to corresponding patients, research on medical beds in various types has been conducted in order to solve these problems.

A representative topic that has been actively discussed in the recent research on medical beds is discrimination of lying postures. Researchers have a common position that study on user's lying posture discrimination is closely related to patient's potential comfort and the prevention of pressure ulcers.

In a case where posture discrimination is successfully implemented, smart medical beds may be of great help in relieving the psychological and physical burden of medical staff and care givers and in providing high-level medical services.

For this reason, many studies are being conducted to predict patient's lying postures through artificial intelligence (AI) algorithms. To summarize recent studies and major achievements of posture discrimination using the artificial intelligence algorithms, kNN clustering as a representative algorithm has shown significant performance in several studies. In a study of related art 1 (Yousefi et al.), pressure was measured by changes in an air tube in order to adjust a bed to fit a patient's body, and a study on discriminating postures by inserting the measured pressure as input values into principal component analysis (PCA) and kNN was conducted.

In addition, in a study of related art 2 (Viriyavit and Somlertlamvanich), a model for predicting patient's lying positions and postures as a preventive measure against patients falling out of bed by implementing a Bayesian network and a neural network was proposed. It is expected that the proposed model will be applied to an early warning system to perform a function of preventing patient falls. According to the authors' conclusions, it may be confirmed that the potential of artificial intelligence algorithms combined with medical beds is promising despite the small number of samples.

In addition, in related art 3 (Davoodnia et al.), a body mass index (BMI) was predicted based on body pressure of a user lying on a medical bed. The need for convergence of deep learning technology and medical bed technology has been emphasized by proving that a multi-task deep learning algorithm having an ensemble structure that combines feature selection of data sets joined in this process is also useful for predicting the patient's body mass index.

As described above, in many related arts, deep learning and machine learning are used to discriminate patient's lying postures. However, despite the efforts of the researchers, there are limitations in terms of the quality and quantity in data sets or participant recruitment used in the experiments of the related arts because a significant portion of time and cost was invested in a data collection process.

### SUMMARY OF THE DISCLOSURE

The present disclosure is devised to solve the above problems, and an objective of the present disclosure is to provide an artificial intelligence-based posture discrimination device using body pressure sensors and a method thereof, wherein, in order to overcome temporal and spatial limitations of experimental research, a Generative Adversarial Network (GAN) for generating data at a level similar to that of actual data using the body pressure sensors without additional data collection work is applied, and based on the data generated through this, an ensemble artificial intelligence deep learning technique combining a Convolutional Neural Network (CNN) with a Long Short-Term Memory (LSTM) neural network model is used, so that user's lying postures are predicted and discriminated, whereby not only an environment capable of indirectly learning potential user's various features generated by age, body shape, etc., which were previously intractable, may be created, but also as the user's lying postures are discriminated more accurately, the function and convenience of pressure ulcer prevention is increased by effectively changing user's body positions.

In order to achieve the objective described above, a first aspect of the present disclosure is to provide an artificial intelligence-based posture discrimination device using body pressure sensors, the device including: a body pressure sensor module installed on an upper part of a frame of a bed or inside a mattress and configured to measure body pressure of a user, touching the frame of the bed or the mattress, by using a plurality of body pressure sensors; a sample body pressure distribution data generation module configured to learn and generate the corresponding user's sample body pressure distribution data by using a Generative Adversarial Network (GAN) preset based on receiving provision of the corresponding user's actual body pressure measurement data measured by the body pressure sensor module; and a posture discrimination module configured to analyze the corresponding user's actual body pressure distribution data on the basis of receiving the provision of the corresponding user's actual body pressure measurement data measured by the body pressure sensor module, and discriminate the corresponding user's lying postures on the basis of learned and analyzed time-series body pressure distribution data in a two-dimensional format after learning and predicting the time-series body pressure distribution data in the two-dimensional format by using an ensemble artificial intelligence deep learning technique combining a Convolutional Neural Network (CNN) and a Long Short-Term Memory (LSTM) neural network model, which have characteristics different from each other, on the basis of the corresponding user's analyzed actual body pressure distribution data and the corresponding user's sample body pressure distribution data generated by the sample body pressure distribution data generation module.

Here, for learning of the Long Short-Term Memory (LSTM) neural network, loss Li may be preferably calculated by Equation 1 below.

$${L}_{i} = - {\sum }_{j} {t}_{i , j} log ( {p}_{i , j} ) \qquad ( Equation 1 )$$

where, p denotes prediction result, t denotes actual data value, i denotes data number, and j denotes class.

Preferably, the corresponding user's lying postures discriminated through the posture discrimination module may include at least one of a supine posture, a recumbent posture on a left side, a recumbent posture on a right side, or a prone posture.

Preferably, the body pressure sensor module may include the plurality of body pressure sensors configured in a three-dimensional structure having time t and a plurality of rows and columns.

Preferably, the artificial intelligence-based posture discrimination device may further include a storage module configured to convert, into a database (DB) for each user, store, and manage user information data of at least one from among the corresponding user's actual body pressure measurement data measured by the body pressure sensor module, the corresponding user's analyzed actual body pressure distribution data, the corresponding user's sample body pressure distribution data generated by the sample body pressure distribution data generation module, the learned and predicted time-series body pressure distribution data in the two-dimensional format, or the corresponding user's lying posture information data discriminated by the posture discrimination module.

Preferably, the artificial intelligence-based posture discrimination device may further include a communication module configured to transmit, to an external terminal or a server in a wired or wireless method, the user information data of at least one from among the corresponding user's actual body pressure measurement data measured by the body pressure sensor module, the corresponding user's analyzed actual body pressure distribution data, the corresponding user's sample body pressure distribution data generated by the sample body pressure distribution data generation module, the learned and predicted time-series body pressure distribution data in the two-dimensional format, or the corresponding user's lying posture information data discriminated by the posture discrimination module.

Preferably, the external terminal or the server may allow the user information data to be displayed on a display screen or to be output as voice, so as to enable a manager to check the user information data of the corresponding user visually or aurally on the basis of receiving provision of the user information data of at least one from among the corresponding user's actual body pressure measurement data, the corresponding user's actual body pressure distribution data, the corresponding user's sample body pressure distribution data, the learned and predicted time-series body pressure distribution data in the two-dimensional format, or the corresponding user's lying posture information data, the user information data being transmitted from the communication module through a pre-installed specific application service.

Preferably, the external terminal or the server may convert, into a database (DB) for each user, store, and manage the user information data of at least one from among the corresponding user's actual body pressure measurement data, the corresponding user's actual body pressure distribution data, the corresponding user's sample body pressure distribution data, the learned and predicted time-series body pressure distribution data in the two-dimensional format, or the corresponding user's lying posture information data, the user information data being transmitted from the communication module through a pre-installed specific application service.

A second aspect of the present disclosure is to provide an artificial intelligence-based posture discrimination method using body pressure sensors, the method using a device provided with a body pressure sensor module, a sample body pressure distribution data generation module, and a posture discrimination module, and including: (a) measuring, by the body pressure sensor module, body pressure of a user touching a frame of a bed or a mattress; (b) learning and generating, by the sample body pressure distribution data generation module, the corresponding user's sample body pressure distribution data by using a Generative Adversarial Network (GAN) preset based on the corresponding user's actual body pressure measurement data measured in step (a); and (c) analyzing, by the posture discrimination module, the corresponding user's actual body pressure distribution data on the basis of the corresponding user's actual body pressure measurement data measured in step (a), and discriminating the corresponding user's lying postures on the basis of learned and predicted time-series body pressure distribution data in a two-dimensional format after learning and predicting the body pressure distribution data in the two-dimensional format in time series by using an ensemble artificial intelligence deep learning technique combining a Convolutional Neural Network (CNN) and a Long Short-Term Memory (LSTM) neural network model, which have characteristics different from each other, on the basis of the corresponding user's analyzed actual body pressure distribution data and the corresponding user's sample body pressure distribution data generated in step (b).

Here, in step (c), for learning of the Long Short-Term Memory (LSTM) neural network, loss Li may be preferably calculated by Equation 1 below.

$${L}_{i} = - {\sum }_{j} {t}_{i , j} log ( {p}_{i , j} ) \qquad ( Equation 1 )$$

where, p denotes prediction result, t denotes actual data value, i denotes data number, and j denotes class.

Preferably, the corresponding user's lying postures discriminated in step (c) may include at least one of a supine posture, a recumbent posture on a left side, a recumbent posture on a right side, or a prone posture.

Preferably, in step (a), the body pressure sensor module may include a plurality of body pressure sensors configured in a three-dimensional structure having time t and a plurality of rows and columns.

Preferably, the artificial intelligence-based posture discrimination method may further include converting, into a database (DB) for each user, by a separate storage module after step (c), storing, and managing user information data of at least one from among the corresponding user's actual body pressure measurement data measured in step (a), the corresponding user's sample body pressure distribution data generated in step (b), the corresponding user's actual body pressure distribution data analyzed in step (c), the time-series body pressure distribution data in the two-dimensional format learned and predicted in step (c), or the corresponding user's lying posture information data discriminated in step (c).

Preferably, the artificial intelligence-based posture discrimination method may further include transmitting, to an external terminal or a server in a wired or wireless method, by a separate communication module after step (c), user information data of at least one from among the corresponding user's actual body pressure measurement data measured in step (a), the corresponding user's sample body pressure distribution data generated in step (b), the corresponding user's actual body pressure distribution data analyzed in step (c), the time-series body pressure distribution data in the two-dimensional format learned and predicted in step (c), or the corresponding user's lying posture information data discriminated in step (c).

Preferably, the external terminal or the server may allow the user information data to be displayed on a display screen or to be output as voice, so as to enable a manager to check the user information data of the corresponding user visually or aurally on the basis of receiving provision of the user information data of at least one from among the corresponding user's actual body pressure measurement data, the corresponding user's actual body pressure distribution data, the corresponding user's sample body pressure distribution data, the learned and predicted time-series body pressure distribution data in the two-dimensional format, or the corresponding user's lying posture information data, the user information data being transmitted through the communication module through a pre-installed specific application service.

Preferably, the external terminal or the server may convert, into a database (DB) for each user, store, and manage the user information data on the basis of receiving the provision of the user information data of at least one from among the corresponding user's actual body pressure measurement data, the corresponding user's actual body pressure distribution data, the corresponding user's sample body pressure distribution data, the learned and predicted time-series body pressure distribution data in the two-dimensional format, or the corresponding user's lying posture information data, the user information data being transmitted through the communication module through a pre-installed specific application service.

A third aspect of the present disclosure is to provide a computer-readable recording medium including a program recorded therein capable of executing, by a computer, the artificial intelligence-based posture discrimination method using the body pressure sensors described above.

The artificial intelligence-based posture discrimination method using the body pressure sensors according to the present disclosure may be implemented as computer-readable code on the computer-readable recording medium. The computer-readable recording medium includes all types of recording devices in which data readable by a computer system is stored.

For example, the computer-readable recording medium includes a ROM, a RAM, a CD-ROM, a magnetic tape, a hard disk, a floppy disk, a removable storage device, a non-volatile memory (i.e., a flash memory), an optical data storage device, etc.

According to the artificial intelligence-based posture discrimination device using the body pressure sensors and the method thereof of the present disclosure as described above, there is provided a strong point that in order to overcome the temporal and spatial limitations of experimental research, the Generative Adversarial Network (GAN) for generating data at the level similar to that of actual data using the body pressure sensors without additional data collection work is applied, and based on the data generated through this, the ensemble artificial intelligence deep learning technique combining the Convolutional Neural Network (CNN) with the Long Short-Term Memory (LSTM) neural network model is used, so that user's lying postures are predicted and discriminated, whereby not only the environment capable of indirectly learning the potential user's various features generated by age, body shape, etc., which were previously intractable, may be created, but also as the user's lying postures are discriminated more accurately, the function and convenience of pressure ulcer prevention is increased by effectively changing user's body positions.

### BRIEF DESCRIPTION OF THE DRAWINGS

FIG. 1 is an overall block configuration view for illustrating an artificial intelligence-based posture discrimination device using body pressure sensors according to an exemplary embodiment of the present disclosure.

FIG. 2 is a conceptual view for illustrating a structure of a Generative Adversarial Network (GAN) applied to the exemplary embodiment of the present disclosure.

FIG. 3 is a view for illustrating result data of the Generative Adversarial Network (GAN) applied to the exemplary embodiment of the present disclosure.

FIG. 4 is a conceptual view for illustrating an ensemble artificial intelligence deep learning structure combining a Convolutional Neural Network (CNN) and a Long Short-Term Memory (LSTM) neural network model, which are applied to the exemplary embodiment of the present disclosure.

FIG. 5 is a detailed block configuration view for illustrating an external terminal applied to the exemplary embodiment of the present disclosure.

FIG. 6 is an overall flowchart for describing an artificial intelligence-based posture discrimination method using body pressure sensors according to the exemplary embodiment of the present disclosure.

FIG. 7 is a view with tables illustrating performance of comparison group classifiers and performance of user's posture shape prediction tested by the artificial intelligence-based posture discrimination method using the body pressure sensors according to the exemplary embodiment of the present disclosure.

### DETAILED DESCRIPTION OF THE DISCLOSURE

The above-described objectives, features, and advantages will be described later in detail with reference to the accompanying drawings, and accordingly, those skilled in the art to which the present disclosure pertains will be able to easily implement the technical idea of the present disclosure. In addition, in describing the present disclosure, when it is determined that a detailed description of a known technology related to the present disclosure may unnecessarily obscure the subject matter of the present disclosure, the detailed description thereof will be omitted.

It will be understood that, although the terms including ordinal numbers, such as first, second, etc. may be used herein to describe various elements, these elements should not be limited by these terms. These terms are only used for the purpose of distinguishing one component from another component. For example, the first component may be referred to as a second component without departing from the scope of the present disclosure, and similarly, the second component may be referred to as a first component. The terminology used herein is for the purpose of describing particular exemplary embodiments only and is not intended to be limiting. As used herein, the singular forms are intended to include the plural forms as well, unless the context clearly indicates otherwise.

The terms used in the present disclosure have selected general terms that are currently widely used as possible while considering functions in the embodiments of the present disclosure, but this may vary according to the intention of a technician working in the field, the judicial precedent, the emergence of new technologies, etc. In addition, in certain cases, there are terms arbitrarily selected by the applicant, and in this case, the meaning of the terms will be described in detail in the description of the corresponding embodiments of the present disclosure. Therefore, the terms used in the present disclosure should be defined based on the meaning of the terms and the overall contents of the present disclosure, not simple names of the terms.

Throughout the description of the present disclosure, when a part is said to “include” or “comprise” a certain component, it means that it may further include or comprise other components, except to exclude other components unless the context clearly indicates otherwise. In addition, terms such as “ . . . unit” and “ . . . module” described in the specification mean units that process at least one function or operation, which may be implemented as hardware or software, or a combination of hardware and software.

Hereinafter, the exemplary embodiment of the present disclosure will be described in detail with reference to the accompanying drawings. It should be understood that the exemplary embodiment of the present disclosure may be changed to a variety of embodiments and the scope and spirit of the present disclosure are not limited to the exemplary embodiment described hereinafter. The embodiments of the present disclosure are provided to more completely describe the present disclosure to those skilled in the art.

Combinations of each block of an attached block diagram and each step of the flowchart may be performed by computer program instructions (i.e., an execution engine), and since these computer program instructions can be loaded on a processor of a general purpose computer, special purpose computer, or other programmable data processing equipment, the instructions, executed by the processor of the computer or other programmable data processing equipment, will produce means for performing the functions described in each block of the attached block diagram or each step of the flowchart. In order to implement functionality in a specific way, these computer program instructions may also be stored in a computer-usable or computer-readable memory capable of supporting a computer or other programmable data processing equipment, so that the instructions stored in the computer-usable or computer-readable memory are able to produce articles of manufacture containing an instruction means for performing functions described in each block in the block diagram or in each step in the flowchart.

In addition, since the computer program instructions may be loaded on the computer or other programmable data processing equipment, a series of operational steps are performed on the computer or other programmable data processing equipment to generate a computer-executed process, so that it is also possible for instructions executing the computer or other programmable data processing equipment to provide steps for executing the functions described in each block of the block diagram and each step of the flowchart.

In addition, it should be noted that each block or each step may represent a module, segment, or part of code that contains one or more executable instructions for executing specified logical functions, and in some alternative embodiments, it is also possible for the functions mentioned in the blocks or steps to be performed out of order. For example, two blocks or steps shown in succession may in fact be performed substantially simultaneously, and the blocks or steps may be performed in the reverse order of the corresponding functions as necessary.

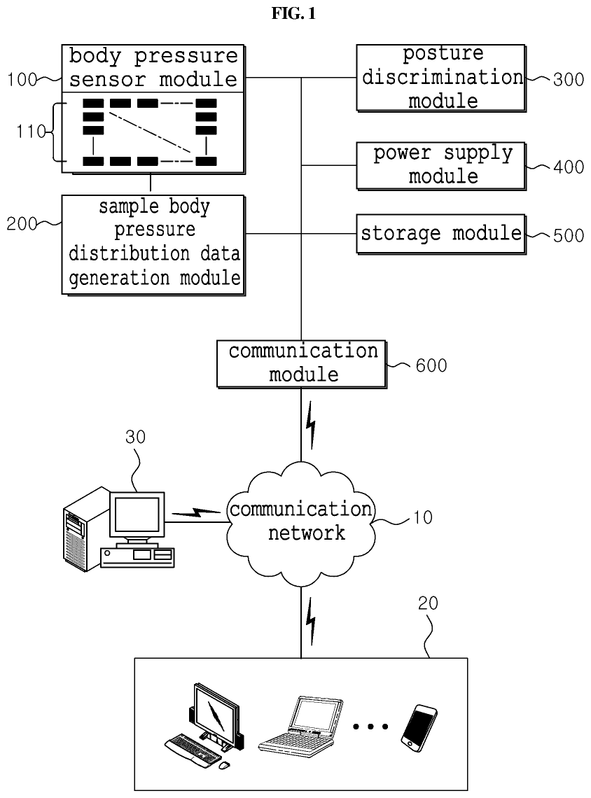

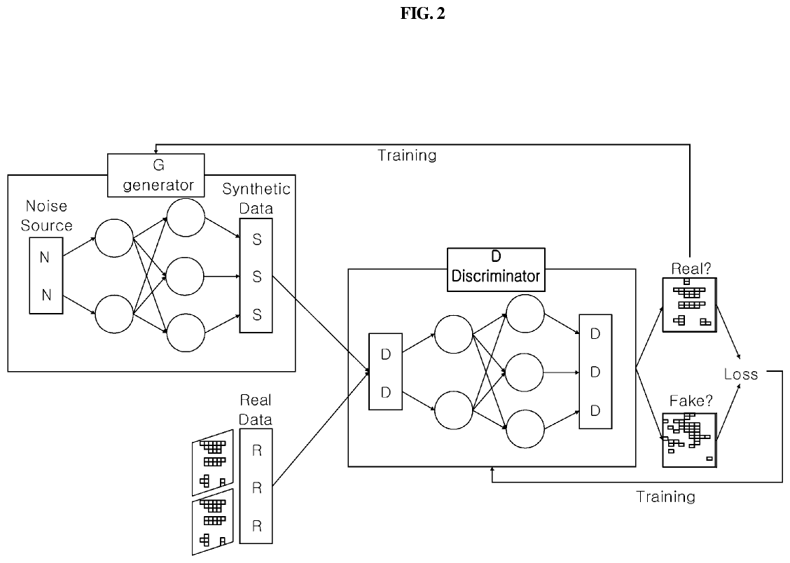

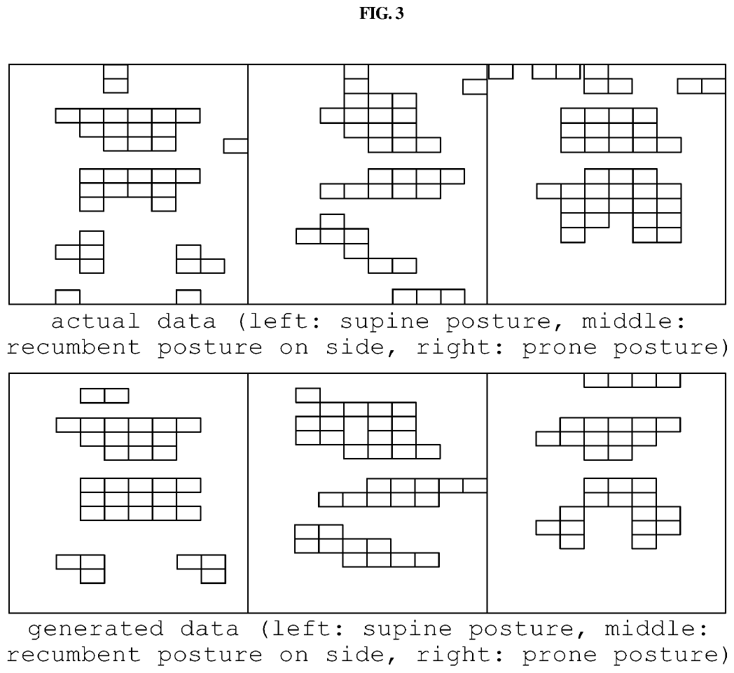

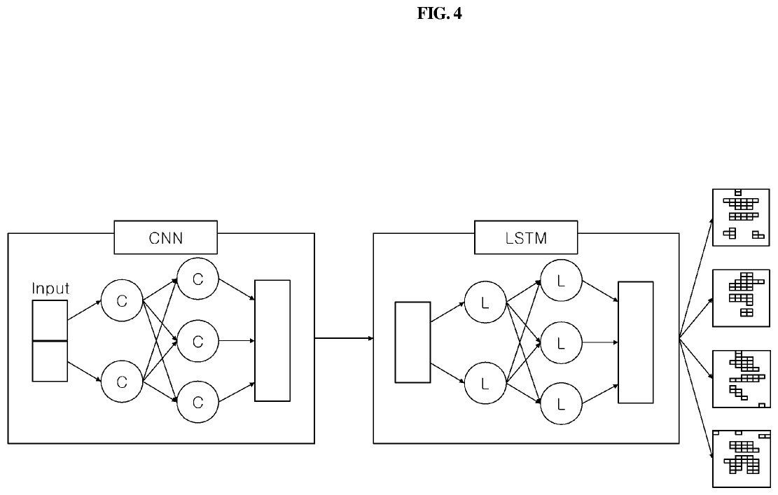

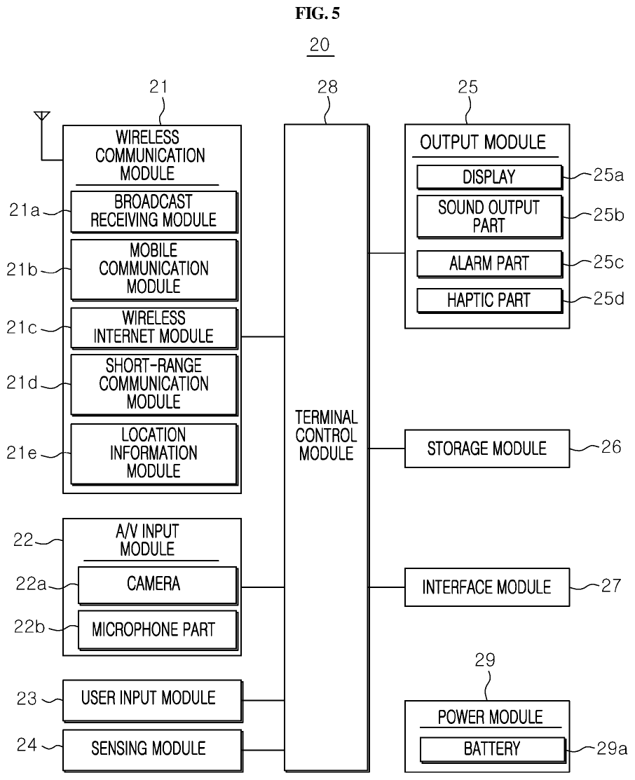

FIG. 1 is an overall block configuration view for illustrating an artificial intelligence-based posture discrimination device using body pressure sensors according to an exemplary embodiment of the present disclosure. FIG. 2 is a conceptual view for illustrating a structure of a Generative Adversarial Network (GAN) applied to the exemplary embodiment of the present disclosure. FIG. 3 is a view for illustrating result data of the Generative Adversarial Network (GAN) applied to the exemplary embodiment of the present disclosure. FIG. 4 is a conceptual view for illustrating an ensemble artificial intelligence deep learning structure combining a Convolutional Neural Network (CNN) and a Long Short-Term Memory (LSTM) neural network model, which are applied to the exemplary embodiment of the present disclosure. FIG. 5 is a detailed block configuration view for illustrating an external terminal applied to the exemplary embodiment of the present disclosure.

Referring to FIGS. 1 to 5, the artificial intelligence-based posture discrimination device using the body pressure sensors according to the exemplary embodiment of the present disclosure is largely configured to include a body pressure sensor module 100, a sample body pressure distribution data generation module 200, a posture discrimination module 300, and a power supply module 400. In addition, the artificial intelligence-based posture discrimination device using the body pressure sensors according to the exemplary embodiment of the present disclosure may additionally include a storage module 500, a communication module 600, an external terminal 20 and/or a server 30, etc. Meanwhile, since the components shown in FIGS. 1 to 5 are not essential, the artificial intelligence-based posture discrimination device using the body pressure sensors according to the exemplary embodiment of the present disclosure may have more components or fewer components than the components shown in FIGS. 1 to 5.

Hereinafter, the components of the artificial intelligence-based posture discrimination device using the body pressure sensors according to the exemplary embodiment of the present disclosure will be described in detail as follows.

The body pressure sensor module 100 is installed on an upper part of a frame of a bed or inside a mattress, and performs, by using a plurality of body pressure sensors 110, a function of measuring body pressure of a user touching the frame of the bed or the mattress.

Preferably, such a body pressure sensor module 100 includes the plurality of body pressure sensors 110 configured in a three-dimensional structure having time t and a plurality of rows and columns.

Here, the body pressure sensor 110 is a type of special purpose pressure sensor for measuring a person's body pressure and distribution thereof, and has been applied to various fields, where a person's movement and a center of gravity are identified and used for posture correction, training, or the like.

For example, the body pressure sensor 110 may be used in a patient bed for preventing or monitoring pressure ulcers of a bedridden patient, a bed for monitoring sleep disorders, a cushion or chair for posture correction, a vehicle seat, an exercise mattress for recognizing changes in the center of gravity in real time, and so on.

In addition, for its purpose, the body pressure sensor 110 unlike other pressure sensors may measure the presence or absence of pressure and the intensity of pressure, and may also express a profile of body pressure on the basis of measuring an intensity of pressure at a specific position.

That is, the body pressure sensor 110 has a uniform sensing characteristic in a plane direction, and is mainly implemented as a film-type body pressure sensor using a polymer resin film or the like.

Such a film-type body pressure sensor uses conductive electrodes disposed on a polymer film having two electrode layers facing each other, and may form both upper and lower flexible printed circuit boards (FPCB s) thereon, form an upper electrode pattern layer and a lower electrode pattern layer respectively on a lower part of the upper flexible printed circuit board and an upper part of the lower flexible printed circuit board, form a piezoresistive layer between the upper electrode pattern layer and the lower electrode pattern layer, combine each layer to form one film substrate structure, and promote user convenience through being flexible.

Meanwhile, unlike the film-type body pressure sensor described above, the body pressure sensor 110 may also be implemented through a substrate-type body pressure sensor. That is, the substrate-type body pressure sensor is formed with a lower electrode configured to include: a pattern part provided with a specific pattern on a hard-type printed circuit board (PCB) and formed in a center of the printed circuit board (PCB); and a connection part extended to a side surface part of the printed circuit board (PCB) for connection with an external circuit. Next, being positioned on the side surface of the printed circuit board (PCB), a contact lead is formed on the connection part of the lower electrode.

In this case, since the contact lead can be easily coupled in a pressing process for bonding the lower substrate and the upper substrate, it is preferable to form the contact lead by using soft lead as a material, and moreover, any material having high conductivity may also be applicable as the material.

In addition, when forming the contact lead described above, heat treatment may also be performed in parallel in order to maximally increase bonding strength. Meanwhile, adjacent to the contact lead, a fixed resistance layer may be additionally formed on the connection part of the lower electrode, so as to stably and uniformly perform a role of a branch resistance connected through an external circuit via the fixed resistance layer, thereby increasing convenience of circuit configuration and the stability and accuracy of measurement.

The sample body pressure distribution data generation module 200 performs a function of learning and generating the corresponding user's sample body pressure distribution data by using a Generative Adversarial Network (GAN) preset based on receiving the corresponding user's actual body pressure measurement data measured by the body pressure sensor module 100.

Here, the Generative Adversarial Network (GAN) is an artificial intelligence algorithm for generating data at a similar level to actual data without additional data collection work, and has been widely used to overcome temporal and spatial limitations of experimental research.

That is, as shown in FIG. 2, in the structure of the Generative Adversarial Network (GAN), a generator and a discriminator operate separately, and data S, generated by the generator, and actual data R is respectively distinguished as fake data and actual data by the discriminator. In a case where the generated data is discriminated as the actual data, the data is used as generator's learning data, and in a case where the generated data is discriminated as the fake data, the data is used as discriminator's learning data. The results of such a Generative Adversarial Network (GAN) is shown in FIG. 3.

The posture discrimination module 300 analyzes the corresponding user's actual body pressure distribution data on the basis of receiving provision of the corresponding user's actual body pressure measurement data measured by the body pressure sensor module, and performs a function of discriminating the corresponding user's lying postures on the basis of the learned and analyzed time-series body pressure distribution data in a two-dimensional format after learning and predicting the time-series body pressure distribution data in the two-dimensional format by using an ensemble artificial intelligence deep learning technique combining a Convolutional Neural Network (CNN) and a Long Short-Term Memory (LSTM) neural network model, which have characteristics different from each other, on the basis of the corresponding user's analyzed actual body pressure distribution data and the corresponding user's sample body pressure distribution data generated by the sample body pressure distribution data generation module 200.

That is, the data generated by over-sampling through the Generative Adversarial Network (GAN) is integrated with existing data previously obtained. The data integrated in this way is used as training data and test data of a Convolutional-Long Short-Tenn Memory (CNN-LSTM) neural network, which is a posture shape prediction algorithm applied in the exemplary embodiment of the present disclosure, and the Convolutional-Long Short-Tenn Memory (CNN-LSTM) neural network is operated as shown in FIG. 4.

Here, for learning of the Long Short-Term Memory (LSTM) neural network, loss Li is preferably calculated by Equation 1 below.

$${L}_{i} = - {\sum }_{j} {t}_{i , j} log ( {p}_{i , j} ) \qquad ( Equation 1 )$$

where, p denotes prediction result, t denotes actual data value, i denotes data number, and j denotes class.

Meanwhile, it is preferable that the corresponding user's lying postures discriminated through the posture discrimination module 300 include, for example, at least one of a supine posture, a recumbent posture on the left side, a recumbent posture on the right side, and/or a prone posture.

In addition, the power supply module 400 performs a function of supplying power required for each module described above, that is, the body pressure sensor module 100, the sample body pressure distribution data generation module 200, the posture discrimination module 300, the storage module 500, the communication module 600, and/or the like. For continuous supplying of the power, it is preferable to implement that a commercial alternating current (AC) power (e.g., AC 220V or 380V) is converted into direct current (DC) and/or alternating current (AC) power, but is not limited thereto and the power supply module 400 may be implemented by including a conventional portable battery as well.

In addition, the power supply module 400 may include a power management unit (not shown) for protecting components from external power shock and outputting a constant voltage. The power management unit may be configured to include an electro static damage (ESD) protector, a power detector, a rectifier, a power circuit breaker, etc.

Here, the ESD protector is configured to protect electronic components from static electricity or sudden power shock. The power detector is configured to transmit a blocking signal to the power circuit breaker when a voltage outside a range of allowable voltage is introduced, and transmit a voltage-raising signal or voltage-lowering signal to the rectifier depending on a voltage change within the range of the allowable voltage. The rectifier is configured to perform a voltage-raising or voltage-lowering rectification operation according to a signal from the power detector so that a constant voltage is supplied by maximally reducing the fluctuation of the input voltage. The power circuit breaker is configured to block the power supplied from the battery according to the blocking signal transmitted from the power detector.

Additionally, the storage module 500 performs a function of converting, into a database (DB) for each user, storing, and managing user information data of at least one from among the corresponding user's actual body pressure measurement data measured by the body pressure sensor module 100, the corresponding user's actual body pressure distribution data analyzed by the posture discrimination module 300, the corresponding user's sample body pressure distribution data generated by the sample body pressure distribution data generation module 200, the time-series body pressure distribution data in the two-dimensional format learned and predicted by the posture discrimination module 300, and/or the corresponding user's lying posture information data discriminated by the posture discrimination module 300.

Such a storage module 500 may include at least one type of storage medium including, for example, a flash memory type memory, a hard disk type memory, a multimedia card micro type memory, a card type memory (e.g., an SD or XD memory, etc.), a Random Access Memory (RAM), a Static Random Access Memory (SRAM), a Read-Only Memory (ROM), an Electrically Erasable Programmable Read-Only Memory (EEPROM), a Programmable Read-Only Memory (PROM), a magnetic memory, a magnetic disk, and an optical disk.

The communication module 600 performs a function of transmitting, to an external terminal 20 and/or server 30 in a wired and/or wireless communication method through the communication network 10, the user information data of at least one from among the corresponding user's actual body pressure measurement data measured by the body pressure sensor module 100, the corresponding user's actual body pressure distribution data analyzed by the posture discrimination module 300, the corresponding user's sample body pressure distribution data generated by the sample body pressure distribution data generation module 200, the time-series body pressure distribution data in the two-dimensional format learned and predicted by the posture discrimination module 300, and/or the corresponding user's lying posture information data discriminated through the posture discrimination module 300.

Such a communication module 600 may also use, for example, short-distance communication methods of at least one from among Bluetooth, ZigBee, Beacon, Ultra-Wideband (UWB), Radio Frequency Identification (RFID), and/or infrared (IR) communication.

Meanwhile, preferably, a communication network 10 is configured to include an Ethernet or mobile communication network, etc. The communication network 10 may be a high-speed backbone network of a large communication network capable of providing large-capacity, long-distance voice and data services, and may be a next-generation wireless network including WiFi, WiGig, Wireless Broadband Internet (WiBro), and World Interoperability for Microwave Access (WiMAX), which are for providing Internet or high-speed multimedia services.

The Internet refers to a worldwide open computer network structure that provides various services present on a TCP/IP protocol and an upper layer of the TCP/IP, the various services including: Hyper Text Transfer Protocol (HTTP), Telnet, File Transfer Protocol (FTP), Domain Name System (DNS), Simple Mail Transfer Protocol (SMTP), Simple Network Management Protocol (SNMP), Network File Service (NFS), Network Information Service (NIS), etc., and provides an environment enabling the communication module 600 to be connected to an external terminal 20 and/or server 30. Meanwhile, the Internet may be a wired or wireless Internet, and may also be a core network integrated with a wired public network, a wireless mobile communication network, a mobile Internet, or the like.

When provided with a mobile communication network, the communication network 10 may be a synchronous mobile communication network or an asynchronous mobile communication network. A Wideband Code Division Multiple Access (WCDMA) communication network may be referred to as an exemplary embodiment of the asynchronous mobile communication network. In this case, although not shown in the drawings, the mobile communication network may include a Radio Network Controller (RNC). Meanwhile, although the WCDMA network is taken as an example, the communication network may be a next-generation communication network such as a 3G LTE network, a 4G network, or a 5G network, or may additionally be an IP network based on IPs. Such a communication network 10 performs a role of mutually transmitting signals and data between the communication module 600 and the external terminal 20 and/or the server 30.

In addition, the external terminal 20 and/or the server 30 performs a function of allowing the user information data to be displayed on a display screen or to be output as voice so that a manager may visually or aurally check the information data of the corresponding user on the basis of receiving provision of the user information data of at least one from among the corresponding user's actual body pressure measurement data, the corresponding user's actual body pressure distribution data, the corresponding user's sample body pressure distribution data, the learned and predicted time-series body pressure distribution data in the two-dimensional format, and/or the corresponding user's lying posture information data, the user information data being transmitted from the communication module through a pre-installed specific application service.

In addition, the external terminal 20 and/or the server 30 may perform the function of converting, into the database (DB) for each user, storing, and managing the user information data of at least one from among the corresponding user's actual body pressure measurement data, the corresponding user's actual body pressure distribution data, the corresponding user's sample body pressure distribution data, the learned and predicted time-series body pressure distribution data in the two-dimensional format, and/or the corresponding user's lying posture information data, the user information data being transmitted from the communication module through the pre-installed specific application service.

Meanwhile, the external terminal 20 described above is preferably configured to include a mobile terminal system of at least one from among a smart phone, a smart pad, and/or a smart note, which communicate through wireless Internet or mobile Internet. In addition, including a palm PC, a mobile play-station, and a Digital Multimedia Broadcasting (DMB) phone having a communication function, a tablet PC, an iPad, etc., the external terminal 20 may be collectively referred to all wired/wireless home appliance/communication systems having a user interface for connecting to the communication module 600.

As shown in FIG. 5, such an external terminal 20 may include a wireless communication module 21, an audio/video (A/V) input module 22, a user input module 23, and a sensing module 24, an output module 25, a storage module 26, an interface module 27, a terminal control module 28, a power module 29, etc. Meanwhile, since the components shown in FIG. 5 are not essential, the external terminal 20 may have more components or fewer components than the components shown in FIG. 5.

Hereinafter, a detailed description of the components of the external terminal 20 is as follows.

The wireless communication module 21 may include one or more modules enabling wireless communication between the external terminal 20 and the communication module 600 and/or the server 30. For example, the wireless communication module 21 may include a broadcast receiving module 21a, a mobile communication module 21b, a wireless Internet module 21c, a short-range communication module 21d, a location information module 21e, etc.

The broadcast receiving module 21a receives broadcast signals (e.g., TV broadcast signals, radio broadcast signals, data broadcast signals, and the like) and/or broadcast-related information from an external broadcast management server through various broadcast channels (e.g., satellite channels, terrestrial channels, and the like).

On a mobile communication network, the mobile communication module 21b transmits and receives a wireless signal with at least one of a base station, an external terminal 20, and a server. The wireless signal may include a voice call signal, a video call signal, or various types of data according to transmission/reception of text/multimedia messages.

The wireless Internet module 21c is a module for accessing the wireless Internet, and may be built in or external to the external terminal 20. As the wireless Internet technology, for example, WLAN (Wi-Fi), WiBro, WiMAX, HSDPA, LTE, and the like may be used.

The short-range communication module 21d is a module for short-range communication, and may use, for example, Bluetooth communication, ZigBee communication, Ultra-Wideband (UWB) communication, Radio Frequency Identification (RFID) communication, or infrared (IR) communication, etc.

The location information module 21e is a module for checking or obtaining a location of the external terminal 20, and may obtain current location information of the external terminal 20 by using a Global Position System (GPS), etc.

Meanwhile, transmitting and receiving data with the server 30 and/or the communication module 600 may be performed by using a specific application program stored in the storage module 26 through the above-described wireless communication module 21 and/or wired communication module (not shown) under the control of the terminal control module 28.

The A/V input module 22 is a module for inputting an audio signal or a video signal, and may fundamentally include a camera 22a, a microphone part 22b, etc. The camera 22a processes image frames such as still images or videos obtained by an image sensor in a video call mode or a photographing mode. The microphone part 22b receives an external sound signal by a microphone in a call mode, a recording mode, or a voice recognition mode, and processes the external sound signal as electrical voice data.

The user input module 23 is a module that generate input data for controlling an operation of the external terminal 20, and in particular, performs a function of inputting a selection signal for any one of pieces of data management information displayed through a display 25a of the output module 25. The selection signal may be input, for example, by using a touch panel (with a static pressure/electricity type) input by a user's touch or by using a separate input device (e.g., a keypad dome switch, a jog wheel, a jog switch, and the like).

The sensing module 24 generates a sensing signal for controlling the operation of the external terminal 20 by detecting a current state of the external terminal 20, the current state including an open/closed state of the external terminal 20, a location of the external terminal 20, presence or absence of user touch, user's action of touching to a specific part, an orientation of the external terminal 20, acceleration/deceleration of the external terminal 20, etc. Such a sensing signal is transmitted to the terminal control module 28, and may serve as a basis for the terminal control module 28 to perform a specific function.

The output module 25 is a module for generating an output related to visual, auditory, or tactile sense, and fundamentally, may include a display 25a, a sound output part 25b, an alarm part 25c, and a haptic part 25d, and the like.

The display 25a is for displaying and outputting information processed by the external terminal 20.

For example, when the external terminal 20 is in a call mode, a User Interface (UI) or a Graphical User Interface (GUI), which are related to a call, is displayed, and when the external terminal 20 is in a video call mode or a photographing mode, a photographed and/or received image, UI, or GUI is displayed on the display 25a.

For example, in a mode such as a call signal reception mode, a call mode or a recording mode, a voice recognition mode, a broadcast reception mode, the sound output part 25b may output audio data, which is stored in the storage module 26 or received from the wireless communication module 21.

The alarm part 25c may output a signal for notifying the occurrence of an event of the external terminal 20. Examples of events occurring in the external terminal 20 include call signal reception, message reception, key signal input, touch input, etc.

The haptic part 25d generates various tactile effects that a user may feel. A typical example of a tactile effect generated by the haptic part 25d is vibration. The intensity, pattern, etc. of the vibration generated by the haptic part 25d is controllable.

The storage module 26 may store a program for an operation of the terminal control module 28, and may temporarily store input/output data (e.g., data related to a phonebook, a message, a still image, a video, and the like).

In addition, the storage module 26 may store data on the vibrations and sounds of various patterns, which are output when inputting a touch on a touch screen, and may store an application program related to specific posture discrimination.

In addition, since the storage module 26 may store source data for generating posture discrimination-related information, posture discrimination-related data may be constructed in a form composed of images and sounds, and a process and result of generating the posture discrimination-related data may also be stored together.

The storage module 26 may include at least one type of storage medium, including a flash memory type storage, a hard disk type storage, a multimedia card micro type storage, a card type memory (e.g., an SD or XD memory, etc.), a RAM, a SRAM, a ROM, an EEPROM, a PROM, a magnetic memory, a magnetic disk, an optical disc, etc.

The interface module 27 serves as a passage for all external devices connected to the external terminal 20. The interface module 27 receives transmitted data or supplied power from an external device, so as to transmit the data or power to each component inside the external terminal 20 or transmit the data inside the external terminal 20 to an external device.

The terminal control module 28 generally controls overall operations of the external terminal 20, and performs related control and processing for the operations such as a voice call, data communication, a video call, and execution of various applications.

That is, the terminal control module 28 performs a control function, so as to control an application program related to posture discrimination stored in the storage module 26 to be executed, make a request for generating posture discrimination-related data through execution of the application program related to the posture discrimination, and receive the posture discrimination-related data for the above request.

In addition, through the execution of the posture discrimination-related application program, the terminal control module 28 performs a control function so that in a process of generating data related to discrimination of postures desired by a user, auxiliary elements including at least one of video, audio, and sound are output to at least one of the display 25a and other output devices (e.g., the sound output part 25b, alarm part 25c, haptic part 25d, and the like).

In addition, the terminal control module 28 may monitor charging current and charging voltage of the battery 29a at all times, and temporarily store the monitored values in the storage module 26. In this case, it is preferable that the storage module 26 stores not only battery charging status information such as the monitored charging current and charging voltage, but also battery specification information (i.e., product codes, ratings, and the like).

The power module 29 receives external power and internal power applied under the control of the terminal control module 28 to supply the power required for the operation of each component. The power module 29 supplies the power from the built-in battery 29a to each component to operate, and is capable of charging the battery by using a charging terminal (not shown).

Various exemplary embodiments described herein may be implemented in a recording medium that can be read by a computer or a similar device by using, for example, software, hardware, or a combination thereof.

According to the hardware implementation, the exemplary embodiment described herein may be implemented by using at least one of electrical units for performing functions of Application Specific Integrated Circuits (ASICs), Digital Signal Processors (DSPs), Digital Signal Processing Devices (DSPDs), Programmable Logic Devices (PLDs), Field Programmable Gate Arrays (FPGAs), processors, controllers, microcontrollers, and microprocessors. In some cases, such exemplary embodiments may be implemented by the terminal control module 28.

According to the software implementation, the exemplary embodiments such as procedures or functions may be implemented together with separate software modules that perform at least one of functions or operations. Software code may be implemented by a software application written in an appropriate programming language. In addition, the software code may be stored in the storage module 26 and executed by the terminal control module 28.

When the external terminal 20 is configured to include a smartphone, unlike ordinary mobile phones (aka feature phones), the smartphone is a phone based on an open operating system that allows a user to download, freely use, and delete various application programs he or she desires, and the smartphone is preferably referred to as a communication device that includes: all mobile phones provided with mobile office functions, as well as functions such as voice/video calls and Internet data communication, which are generally used; or any Internet phone or a tablet PC that does not have a voice call function but is capable of accessing the Internet.

As described above, since the smartphone uses the open operating system, unlike a mobile phone having a closed operating system, the user is able to arbitrarily install and manage various application programs.

Hereinafter, an artificial intelligence-based posture discrimination method using body pressure sensors according to the exemplary embodiment of the present disclosure will be described in detail.

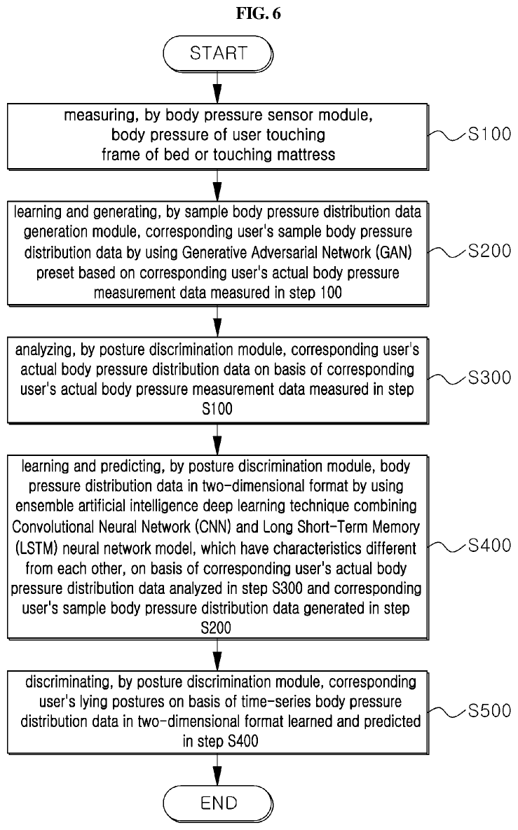

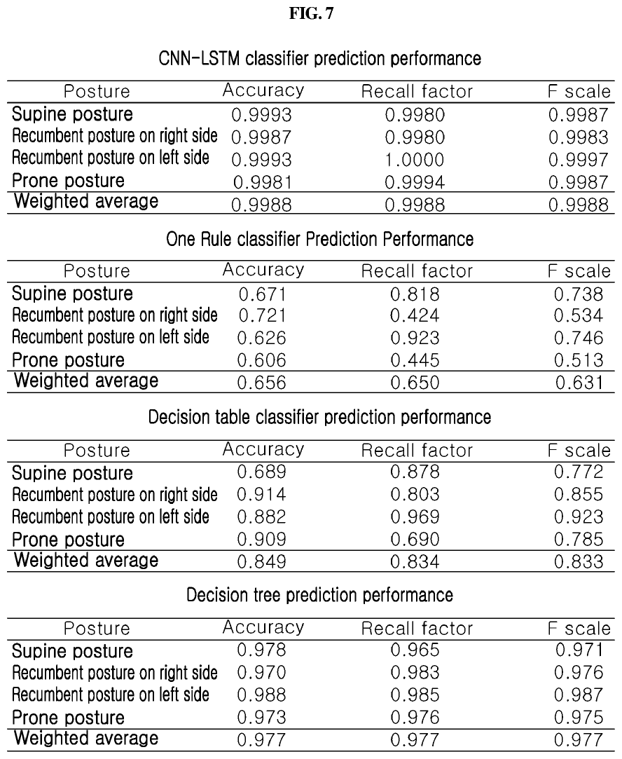

FIG. 6 is an overall flowchart for describing an artificial intelligence-based posture discrimination method using body pressure sensors according to the exemplary embodiment of the present disclosure. FIG. 7 is a view with tables illustrating performance of comparison group classifiers and performance of user's posture shape prediction tested by the artificial intelligence-based posture discrimination method using the body pressure sensors according to the exemplary embodiment of the present disclosure.

Referring to FIGS. 1 to 7, in step S100, first, the artificial intelligence-based posture discrimination method using the body pressure sensors according to the exemplary embodiment of the present disclosure measures body pressure of a user touching a frame of a bed or touching a mattress through a body pressure sensor module 100.

In this case, in step S100 described above, the body pressure sensor module 100 is preferably configured such that a plurality of body pressure sensors 110 installed on an upper part of the frame of the bed or inside the mattress has a three-dimensional structure having time t and a plurality of rows and columns.

Thereafter, in step S200, the corresponding user's sample body pressure distribution data is learned and generated, by a sample body pressure distribution data generation module 200, by using a Generative Adversarial Network (GAN) preset based on the corresponding user's actual body pressure measurement data measured in step 100.

Next, in step S300, the corresponding user's actual body pressure distribution data is analyzed based on the corresponding user's actual body pressure measurement data measured in step S100 by a posture discrimination module 300.

Next, in step S400, body pressure distribution data in a two-dimensional format is learned and predicted, by the posture discrimination module 300, by using an ensemble artificial intelligence deep learning technique combining a Convolutional Neural Network (CNN) and a Long Short-Term Memory (LSTM) neural network model, which have characteristics different from each other, on the basis of the corresponding user's actual body pressure distribution data analyzed in step S300 and the corresponding user's sample body pressure distribution data generated in step S200.

In this case, in step S400, for learning of the Long Short-Term Memory (LSTM) neural network, loss Li is preferably calculated by Equation 1 below.

$${L}_{i} = - {\sum }_{j} {t}_{i , j} log ( {p}_{i , j} ) \qquad ( Equation 1 )$$

where, p denotes prediction result, t denotes actual data value, i denotes data number, and j denotes class.

Thereafter, in step S500, the corresponding user's lying postures are discriminated, by the posture discrimination module 300, on the basis of the time-series body pressure distribution data in the two-dimensional format learned and predicted in step S400.

In this case, preferably, the corresponding user's lying postures discriminated in step S500 include, for example, at least one posture from among a supine posture, a recumbent posture on the left side, a recumbent posture on the right side, and/or a prone posture.

Additionally, although not shown in the drawing, after step S500, the artificial intelligence-based posture discrimination method may further include converting, into a database (DB) for each user in a separate storage module 500, storing, and managing user information data of at least one from among the corresponding user's actual body pressure measurement data measured in step S100, the corresponding user's sample body pressure distribution data generated in step S200, the corresponding user's actual body pressure distribution data analyzed in step S300, the time-series body pressure distribution data in the two-dimensional format learned and predicted in step S400, and/or the corresponding user's lying posture information data discriminated in step S500.

In addition, after step S500, the artificial intelligence-based posture discrimination method may further include transmitting, to an external terminal 20 and/or a server 300 in a wired and/or wireless communication method through a separate communication module 600, the user information data of at least one from among the corresponding user's actual body pressure measurement data measured in step S100, the corresponding user's sample body pressure distribution data generated in step S200, the corresponding user's actual body pressure distribution data analyzed in step S300, the time-series body pressure distribution data in the two-dimensional format learned and predicted in step S400, and/or the corresponding user's lying posture information data discriminated in step S500.

In addition, the external terminal 20 and/or the server 30 may allow the user information data to be displayed on a display screen or to be output as voice so that a manager may visually or aurally check the user information data of the corresponding user on the basis of receiving provision of the user information data of at least one from among the corresponding user's actual body pressure measurement data, the corresponding user's actual body pressure distribution data, the corresponding user's sample body pressure distribution data, the learned and predicted time-series body pressure distribution data in the two-dimensional format, and/or the corresponding user's lying posture information data, the user information data being transmitted by the communication module 600 through a pre-installed specific application service.

In addition, the external terminal 20 and/or the server 30 may convert, into the database (DB) for each user, store, and manage the user information data on the basis of receiving the provision of the user information data of at least one from among the corresponding user's actual body pressure measurement data, the corresponding user's actual body pressure distribution data, the corresponding user's sample body pressure distribution data, the learned and predicted time-series body pressure distribution data in the two-dimensional format, and/or the corresponding user's lying posture information data, the user information data being transmitted by the communication module through the pre-installed specific application service.

In order to confirm the effective results of the artificial intelligence-based posture discrimination method using the body pressure sensors according to the exemplary embodiment of the present disclosure described above, the body pressure data for each posture were collected from 13 subjects having various biological and physical features.

In addition, the entire process and programming of the corresponding data collection and preprocessing were performed with Python 3.7 in a Windows 10 operating system environment. The artificial intelligence models applied to the posture discrimination are (1) the Convolutional Neural Network (CNN), and (2) the Long Short-Term Memory (LSTM) neural network, and in relation to data generation, (3) the Generative Adversarial Network (GAN) was applied thereto.

In particular, by combining two artificial intelligence neural networks having different characteristics, the body pressure distribution (image) data in the two-dimensional format is accumulated and analyzed effectively. The combined ensemble model is called the Convolutional-Long Short-Term Memory (CNN-LSTM) for short, and the results derived through the Convolutional-Long Short-Term Memory (CNN-LSTM) are compared with results of other models and previous studies.

In addition, “categorical crossentropy” is applied to the loss function of the experimental results. Accuracy, recall factors, and F scales are used to evaluate the experimental results. FIG. 7 shows the posture shape prediction performance of the Convolutional-Long Short-Term Memory (CNN-LSTM) neural network and the performance of comparison group classifiers (i.e., a One Rule classifier, a decision table classifier, and a decision tree classifier).

That is, the performance of the classifiers was confirmed in the order of the Convolutional-Long Short-Term Memory (CNN-LSTM) neural network>the decision tree>the decision table>the One Rule. In particular, an accuracy value of 99.88% calculated as a weighted average was found to be superior to accuracy of initial levels confirmed in previous studies.

In conclusion, in the present disclosure, the experiment was conducted to discriminate the patient's lying positions on the basis of the body pressure sensors in order to confirm the posture prediction performance of the artificial intelligence algorithm to be installed in medical beds.

For this experiment, the medical bed equipped with the body pressure sensors on the bed mattress composed in a structure of 17 keyboards was used as a core artifact, and the data was obtained from 13 subjects. The posture discrimination method using artificial intelligence operated based on the body pressure data has been proposed on the basis of diversifying the samples by applying the Generative Adversarial Network (GAN) to the obtained data.

That is, in order to confirm the prediction performance of the present disclosure, the improvement in terms of quantitative and qualitative aspects of the data to be used in the present disclosure was achieved by using the Generative Adversarial Network (GAN) based on the Convolutional Neural Network (CNN) on the basis of collecting the fundamental data by having 13 subjects with various biological and physical features assume four types of postures.

By applying the above Generative Adversarial Network (GAN), the environment in which it is possible to indirectly learn potential users' various features caused by age, body shape, etc., which were previously intractable, was created. Thereafter, the Convolutional-Long Short-Term Memory (CNN-LSTM) algorithm, which learns and predicts image-type input values in time series, was applied to discriminate the users' lying postures.

As a result, it was confirmed that the posture discrimination performance of the proposed Convolutional-Long Short-Term Memory (CNN-LSTM) was found to be superior to the prediction results of other benchmark algorithms. Furthermore, excellent performance was achieved when compared with the prediction performance of previous studies.

According to the present disclosure, it was confirmed that the Convolutional-Long Short-Term Memory (CNN-LSTM) is the algorithm suitable for the posture discrimination. Therefore, it is expected that this Convolutional-Long Short-Term Memory (CNN-LSTM) algorithm may be combined with medical beds in the future and be applicable as a part of the pressure ulcer prevention algorithm.

As described above, by collecting the data from the subjects having various physical features, and by generating the samples using the Generative Adversarial Network (GAN), the present disclosure attempts to overcome technical limitations caused by the lack of samples. In addition, the body pressure sensors having the time-series image features were effectively analyzed by applying the Convolutional-Long Short-Term Memory (CNN-LSTM) model combining two deep learning algorithms.

Meanwhile, the artificial intelligence-based posture discrimination method using the body pressure sensors according to the exemplary embodiment of the present disclosure can be implemented as computer-readable code on a computer-readable recording medium. The computer-readable recording medium includes all types of recording devices in which data readable by a computer system is stored.

For example, the computer-readable recording medium includes a ROM, a RAM, a CD-ROM, a magnetic tape, a hard disk, a floppy disk, a removable storage device, a non-volatile memory (i.e., a flash memory), an optical data storage device, etc.

In addition, the computer-readable recording medium may be distributed to computer systems connected through a computer communication network, so as to be stored and executed as the readable code in a distributed manner.

The preferred exemplary embodiment of the artificial intelligence-based posture discrimination device using the body pressure sensors and the method thereof according to the present disclosure described above has been described, but is not limited thereto, and it is possible to embody the present disclosure with various modifications within the scope of the claims, the detailed description of the disclosure, and the accompanying drawings, and this embodiment also belongs to the present disclosure.

1. An artificial intelligence-based posture discrimination device using body pressure sensors, the device comprising:
a body pressure sensor module installed on an upper part of a frame of a bed or inside a mattress and configured to measure body pressure of a user, touching the frame of the bed or the mattress, by using a plurality of body pressure sensors;
a sample body pressure distribution data generation module configured to learn and generate the corresponding user's sample body pressure distribution data by using a Generative Adversarial Network (GAN) preset based on receiving provision of the corresponding user's actual body pressure measurement data measured by the body pressure sensor module; and
a posture discrimination module configured to analyze the corresponding user's actual body pressure distribution data on the basis of receiving the provision of the corresponding user's actual body pressure measurement data measured by the body pressure sensor module, and discriminate the corresponding user's lying postures on the basis of learned and analyzed time-series body pressure distribution data in a two-dimensional format after learning and predicting the time-series body pressure distribution data in the two-dimensional format by using an ensemble artificial intelligence deep learning technique combining a Convolutional Neural Network (CNN) and a Long Short-Term Memory (LSTM) neural network model, which have characteristics different from each other, on the basis of the corresponding user's analyzed actual body pressure distribution data and the corresponding user's sample body pressure distribution data generated by the sample body pressure distribution data generation module.

2. The device of claim 1, wherein, for learning of the Long Short-Term Memory (LSTM) neural network, loss (Li) is calculated by Equation 1 below:
$${L}_{i} = - {\sum }_{j} {t}_{i , j} log ( {p}_{i , j} ) \qquad ( Equation 1 )$$
where, p denotes prediction result, t denotes actual data value, i denotes data number, and j denotes class.

3. The device of claim 1, wherein the corresponding user's lying postures discriminated through the posture discrimination module comprises at least one of a supine posture, a recumbent posture on a left side, a recumbent posture on a right side, or a prone posture.

4. The device of claim 1, wherein the body pressure sensor module comprises the plurality of body pressure sensors configured in a three-dimensional structure having time (t) and a plurality of rows and columns.

5. The device of claim 1, further comprising:
a storage module configured to convert, into a database (DB) for each user, store, and manage user information data of at least one from among the corresponding user's actual body pressure measurement data measured by the body pressure sensor module, the corresponding user's analyzed actual body pressure distribution data, the corresponding user's sample body pressure distribution data generated by the sample body pressure distribution data generation module, the learned and predicted time-series body pressure distribution data in the two-dimensional format, or the corresponding user's lying posture information data discriminated by the posture discrimination module.

6. The device of claim 1, further comprising:
a communication module configured to transmit, to an external terminal or a server in a wired or wireless method, the user information data of at least one from among the corresponding user's actual body pressure measurement data measured by the body pressure sensor module, the corresponding user's analyzed actual body pressure distribution data, the corresponding user's sample body pressure distribution data generated by the sample body pressure distribution data generation module, the learned and predicted time-series body pressure distribution data in the two-dimensional format, or the corresponding user's lying posture information data discriminated by the posture discrimination module.

7. The device of claim 6, wherein the external terminal or the server allows the user information data to be displayed on a display screen or to be output as voice, so as to enable a manager to check the user information data of the corresponding user visually or aurally on the basis of receiving provision of the user information data of at least one from among the corresponding user's actual body pressure measurement data, the corresponding user's actual body pressure distribution data, the corresponding user's sample body pressure distribution data, the learned and predicted time-series body pressure distribution data in the two-dimensional format, or the corresponding user's lying posture information data, the user information data being transmitted from the communication module through a pre-installed specific application service.

8. The device of claim 6, wherein the external terminal or the server converts, into a database (DB) for each user, stores, and manages the user information data of at least one from among the corresponding user's actual body pressure measurement data, the corresponding user's actual body pressure distribution data, the corresponding user's sample body pressure distribution data, the learned and predicted time-series body pressure distribution data in the two-dimensional format, or the corresponding user's lying posture information data, the user information data being transmitted from the communication module through a pre-installed specific application service.

9. An artificial intelligence-based posture discrimination method using body pressure sensors, the method using a device provided with a body pressure sensor module, a sample body pressure distribution data generation module, and a posture discrimination module, and comprising:
(a) measuring, by the body pressure sensor module, body pressure of a user touching a frame of a bed or a mattress;
(b) learning and generating, by the sample body pressure distribution data generation module, the corresponding user's sample body pressure distribution data by using a Generative Adversarial Network (GAN) preset based on the corresponding user's actual body pressure measurement data measured in step (a); and
(c) analyzing, by the posture discrimination module, the corresponding user's actual body pressure distribution data on the basis of the corresponding user's actual body pressure measurement data measured in step (a), and discriminating the corresponding user's lying postures on the basis of learned and predicted time-series body pressure distribution data in a two-dimensional format after learning and predicting the body pressure distribution data in the two-dimensional format in time series by using an ensemble artificial intelligence deep learning technique combining a Convolutional Neural Network (CNN) and a Long Short-Term Memory (LSTM) neural network model, which have characteristics different from each other, on the basis of the corresponding user's analyzed actual body pressure distribution data and the corresponding user's sample body pressure distribution data generated in step (b).

10. The method of claim 9, wherein, in step (c), for learning of the Long Short-Term Memory (LSTM) neural network, loss (Li) is calculated by Equation 1 below:
$${L}_{i} = - {\sum }_{j} {t}_{i , j} log ( {p}_{i , j} ) \qquad ( Equation 1 )$$
where, p denotes prediction result, t denotes actual data value, i denotes data number, and j denotes class.

11. The method of claim 9, wherein the corresponding user's lying postures discriminated in step (c) comprises at least one of a supine posture, a recumbent posture on a left side, a recumbent posture on a right side, or a prone posture.

12. The method of claim 9, wherein, in step (a), the body pressure sensor module comprises a plurality of body pressure sensors configured in a three-dimensional structure having time (t) and a plurality of rows and columns.

13. The method of claim 9, further comprising:
converting, into a database (DB) for each user, by a separate storage module after step (c), storing, and managing user information data of at least one from among the corresponding user's actual body pressure measurement data measured in step (a), the corresponding user's sample body pressure distribution data generated in step (b), the corresponding user's actual body pressure distribution data analyzed in step (c), the time-series body pressure distribution data in the two-dimensional format learned and predicted in step (c), or the corresponding user's lying posture information data discriminated in step (c).

14. The method of claim 9, further comprising:
transmitting, to an external terminal or a server in a wired or wireless method, by a separate communication module after step (c), user information data of at least one from among the corresponding user's actual body pressure measurement data measured in step (a), the corresponding user's sample body pressure distribution data generated in step (b), the corresponding user's actual body pressure distribution data analyzed in step (c), the time-series body pressure distribution data in the two-dimensional format learned and predicted in step (c), or the corresponding user's lying posture information data discriminated in step (c).

15. The method of claim 14, wherein the external terminal or the server allows the user information data to be displayed on a display screen or to be output as voice, so as to enable a manager to check the user information data of the corresponding user visually or aurally on the basis of receiving provision of the user information data of at least one from among the corresponding user's actual body pressure measurement data, the corresponding user's actual body pressure distribution data, the corresponding user's sample body pressure distribution data, the learned and predicted time-series body pressure distribution data in the two-dimensional format, or the corresponding user's lying posture information data, the user information data being transmitted through the communication module through a pre-installed specific application service.

16. The method of claim 14, wherein the external terminal or the server converts, into a database (DB) for each user, stores, and manages the user information data on the basis of receiving the provision of the user information data of at least one from among the corresponding user's actual body pressure measurement data, the corresponding user's actual body pressure distribution data, the corresponding user's sample body pressure distribution data, the learned and predicted time-series body pressure distribution data in the two-dimensional format, or the corresponding user's lying posture information data, the user information data being transmitted through the communication module through a pre-installed specific application service.

17. A computer-readable recording medium comprising:
a program recorded therein capable of executing, by a computer, the method of claim 9.
